(19) United States
(12) Patent Application Publication
(10) Pub. No.: US 2011/0230844 A1
(43) Pub. Date: Sep. 22, 2011

(54) Glass Syringe with Retractable Needle

(76) Inventors: Thomas J. Shaw, Frisco, TX (US); Steven R. Wisner, Frisco, TX (US); Ni Zhu, Plano, TX (US); Mark Small, Leonard, TX (US)

(21) Appl. No.: 13/130,802

(22) PCT Filed: Nov. 23, 2009
(86) PCT No.: PCT/US09/65540

§ 371 Date: May 24, 2011

Related U.S. Application Data
(60) Provisional application No. 61/117,849, filed on Nov. 25, 2008.

(51) Int. Cl. A61M 5/32

(52) U.S. Cl. 604/198

(57) ABSTRACT

A syringe, particularly preferred for use as a prefilled syringe, that has a retractable needle and is characterized by a liquid containment chamber of variable volume, the liquid containment chamber being further defined by surfaces made of glass or an elastomeric material, and not of plastic, and the syringe having no glass part directly contacting another glass part.

### BACKGROUND OF THE INVENTION

1. Field of the Invention

This invention is a syringe designed for use with compositions that would typically require the use of chemically nonreactive glass or rubbery polymers, and further relates to a single-use syringe having a retractable needle that is also suitable for use as a prefilled syringe and that protects any fluid disposed inside the syringe from being degraded by contaminants that leach or migrate into the fluid-containment portion of the syringe during storage or use. The subject syringe preferably has a glass needle holder and barrel, but has no plastic component that contacts fluid contained in the syringe and has no glass parts that directly contact each other.

2. Description of Related Art

During recent decades, plastics have displaced glass as the material of choice for making most of the syringes used throughout the world. The relatively low cost of mass-producing molded syringe parts from thermoplastic resins has driven the move from glass to plastic, coupled with increased emphasis upon disposability following a single use as a principal way of controlling the spread of infections such as HIV and other blood-borne pathogens. More recently, plastic syringes having retractable needles have also become widely recognized and available as a preferred technology for reducing the likelihood of accidental needle sticks and the related exposure of healthcare workers and patients to dangerous infectious diseases.

Although some syringes with glass barrels are still commercially available, primarily for use in prefill applications, syringes having glass barrels are typically not designed in such manner that the user can retract the needle following use, and the users of such syringes are, therefore, more susceptible to needlestick injuries and the associated likelihood of pathogenic contamination. Syringe designs that require the use of glass components having close tolerances can be difficult to mass produce. Prior art syringes that utilize glass barrels in combination with one or more other glass parts are also more prone to accidental breakage during handling prior to use if the syringes are designed in such manner that glass parts are allowed to contact each other.

A disadvantage that has been experienced in the use of plastic syringes is the tendency of some medicines or other therapeutic treatment fluids contained inside the syringes to become degraded or contaminated over time. This tendency is of particular concern with prefilled syringes, in which such fluids are commonly stored for prolonged periods such as months or years prior to use. During prolonged storage, substituents or other components of the plastic can leach into the contained fluid, or reactive components in the contained fluid can attack or react with components of the plastic, or gas molecules can migrate through the plastic into the contained fluid, thereby causing oxidation or other degradation. Such degradation can adversely affect the purity, properties and/or therapeutic effectiveness of a fluid before it is administered to a patient, and in some cases, can even render the fluid toxic to patients.

Accordingly, a single use syringe is needed that has a retractable needle but has no fluid-contacting containment surfaces made of plastic or another material susceptible to leaching or migration and no glass components in direct contact with other glass components.

### SUMMARY OF THE INVENTION

The single use syringe disclosed herein comprises a retractable needle and is particularly preferred for use in prefilled applications with fluids that can interact with plastic syringe parts or with fluids that are subject to degradation by migration of gas molecules through the barrel during prolonged storage prior to use. According to one embodiment of the invention, the subject syringe comprises a glass barrel and a needle retraction mechanism having a glass needle holder that cooperate with an elastomeric plunger seal, plunger plug and friction ring to define a non-plastic liquid containment chamber of variable volume, with the volume of the containment chamber depending primarily upon the position of the plunger relative to the barrel. According to another embodiment of the invention, at least one sleeve made of a material other than plastic, most preferably stainless steel, is provided to prevent the liquid from contacting a plastic portion of the plunger handle prior to retraction.

As used herein, the term “glass” can also include other similarly non-reactive materials suitable for use in a pharmaceutical grade application that would normally require glass.

As a general matter, the term “plastic” can include both thermoplastic and thermosetting polymers. Thermoplastic polymers can be resoftened to their original condition by heat; thermosetting polymers cannot. As used herein, the term “plastic” refers primarily to moldable thermoplastic high polymers such as, for example, polyethylene and polypropylene, or an acrylic resin, that also typically contain other ingredients such as curatives, fillers, reinforcing agents, colorants, and/or plasticizers, etc., and that can be formed or molded under heat and pressure. As used herein, the term “plastic” does not include either glass or rubbery elastomers that are approved for use in applications where they are in direct contact with therapeutic liquids that can interact with plastic or that can be degraded by substituents that could otherwise enter the liquid from plastic.

As used herein, the term “elastomeric” or “elastomeric material” refers primarily to crosslinked thermosetting rubbery polymers that are more easily deformable than plastics but that are approved for use with pharmaceutical grade fluids and are not readily susceptible to leaching or gas migration.

As used herein, the term “fluid” refers primarily to liquids, but can also include suspensions of solids dispersed in liquids, and gasses dissolved in or otherwise present together within liquids inside the fluid-containing portions of syringes.

Another significant feature of the present invention is that it has no parts in glass-to-glass contact and also has no plastic parts that contact a liquid disposed inside the syringe before the needle is retracted following use.

It should also be understood and appreciated that the apparatus of the invention, while shown in the accompanying drawings without a needle cover, will be provided with a removable protective needle cover prior to packaging for shipment and storage.

### BRIEF DESCRIPTION OF THE DRAWINGS

The apparatus of the invention is further described and explained in relation to the following drawings wherein:

FIG. 1 is a front elevation view of an embodiment of the syringe of the invention;

FIG. 2 is a left side elevation view of the syringe of FIG. 1;

FIG. 3 is a right side elevation view of the syringe of FIG. 1;

FIG. 4 is an exploded front perspective view of the syringe of claim 1;

FIG. 5 is an enlarged cross-sectional elevation view of a syringe like that of FIG. 1 with the plunger partially depressed inside the glass barrel;

FIG. 6 is an enlarged, cross-sectional elevation view of the syringe of FIG. 5 with the needle fully retracted;

FIG. 7 is an enlarged front elevation view of an embodiment of the barrel of the syringe of FIG. 4;

FIG. 8 is an enlarged front elevation view of an embodiment of the tip cap of the syringe of FIG. 4;

FIG. 9 is an enlarged front elevation view of an embodiment of the glass needle holder of the syringe of FIG. 4;

FIG. 10 is an enlarged cross-sectional front elevation view of an embodiment of the plunger handle of the syringe of FIG. 4;

FIG. 11 is an enlarged cross-sectional front elevation view, partially in section and partially broken away, of an embodiment of the plunger seal of the syringe of FIG. 4;

FIG. 12 is an enlarged front elevation view, partially in section and partially broken away, of the plunger plug of an embodiment of the syringe of FIG. 4;

FIG. 13 is an enlarged cross-sectional front elevation view taken along line 13-13 of FIG. 12;

FIG. 14 is an enlarged front elevation view, partially in section and partially broken away, of an embodiment of the plunger cap of the syringe of FIG. 4;

FIG. 15 is an enlarged cross-sectional front elevation view of an embodiment of the barrel grip of the syringe of FIG. 4;

FIG. 16 is an enlarged front elevation view of an embodiment of the friction ring of the syringe of FIG. 4;

FIG. 17 is an enlarged front elevation view, partially in section and partially broken away, of an embodiment of the friction ring of the syringe of FIG. 4;

FIG. 18 is an enlarged front elevation view, partially in section and partially broken away, of an embodiment of the stainless steel plunger sleeve suitable for use in the syringe of FIG. 4;

FIG. 19 is an enlarged front elevation view, partially in section and partially broken away, of another embodiment of the stainless steel plunger sleeve suitable for use in the syringe of FIG. 4;

FIG. 20 is an enlarged front elevation view, partially in section and partially broken away, of another embodiment of the stainless steel plunger sleeve suitable for use in the syringe of FIG. 4;

FIG. 21 is an enlarged front elevation view of an embodiment of the stainless steel barrel sleeve of the syringe of FIG. 4;

FIG. 22 is an enlarged cross-sectional front elevation view taken along line 22-22 of FIG. 21;

FIG. 23 is an enlarged cross-sectional front elevation view of another embodiment of the syringe of the invention with the plunger shown in a partially depressed position inside the glass barrel;

FIG. 24 is an enlarged cross-sectional front elevation view of the syringe of FIG. 23 with the plunger, plunger plug and friction ring depicted near the positions where they are disposed when retraction is initiated and when the force of the compressed spring is released to drive the needle holder, needle holder cap, needle and displaced plunger plug rearwardly into the retraction cavity inside the plunger;

FIG. 25 is an enlarged cross-sectional front elevation view of the syringe of FIG. 23 following retraction;

FIG. 26 is an enlarged cross-sectional front elevation view of the front end of another embodiment of the syringe of the invention (shown subsequent to retraction) wherein a portion of the plunger seal disposed inside the front end of the plunger provides an engagement surface for the plunger plug; and

FIG. 27 is an enlarged cross-sectional front elevation view of the rear end of another embodiment of the syringe of the invention wherein the rear end of the plunger is closed except for the optional provision of a vent hole through the plunger sidewall.

### DESCRIPTION OF THE PREFERRED EMBODIMENTS

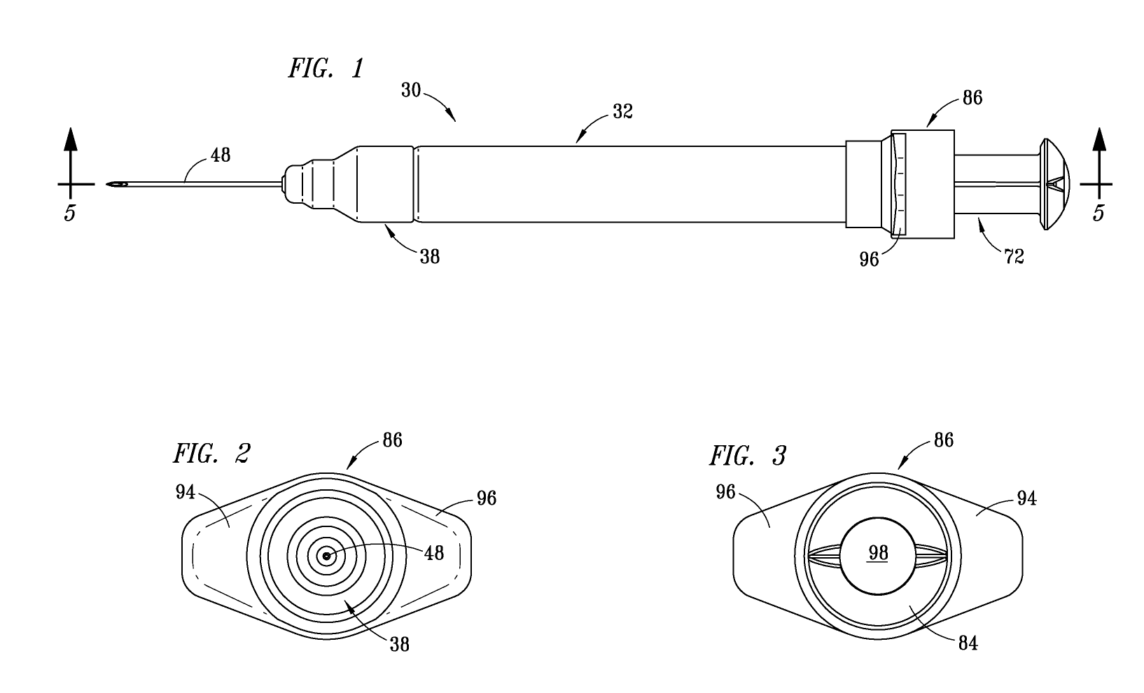

Referring to FIGS. 1-3, syringe 30 of the invention comprises barrel 32, barrel tip cap 38, needle 48, plunger 72 and barrel grip 86. Barrel grip 86 preferably has oppositely disposed, outwardly projecting finger grips 94, 96. Plunger end cap 98 is disposed at the rear of plunger 72 and frictionally engages an opening in the rear end of the plunger. Plunger end cap 98 is preferably vented, to allow air to escape a retraction cavity disposed inside plunger 72 during retraction of needle 48 following use of syringe 32. Barrel 32 is preferably made of “glass” and the portions of plunger 72 that are visible in FIGS. 1-3 are preferably made of “plastic,” as those terms are defined above and are discussed in greater detail below. Plunger grip 86 and barrel tip cap 38 are preferably also made of plastic. It should be understood that any moldable, commercially available plastic that is suitable for use in the intended application can be used in making syringe 30 of the invention, provided that such plastic has all necessary regulatory approvals required for such devices.

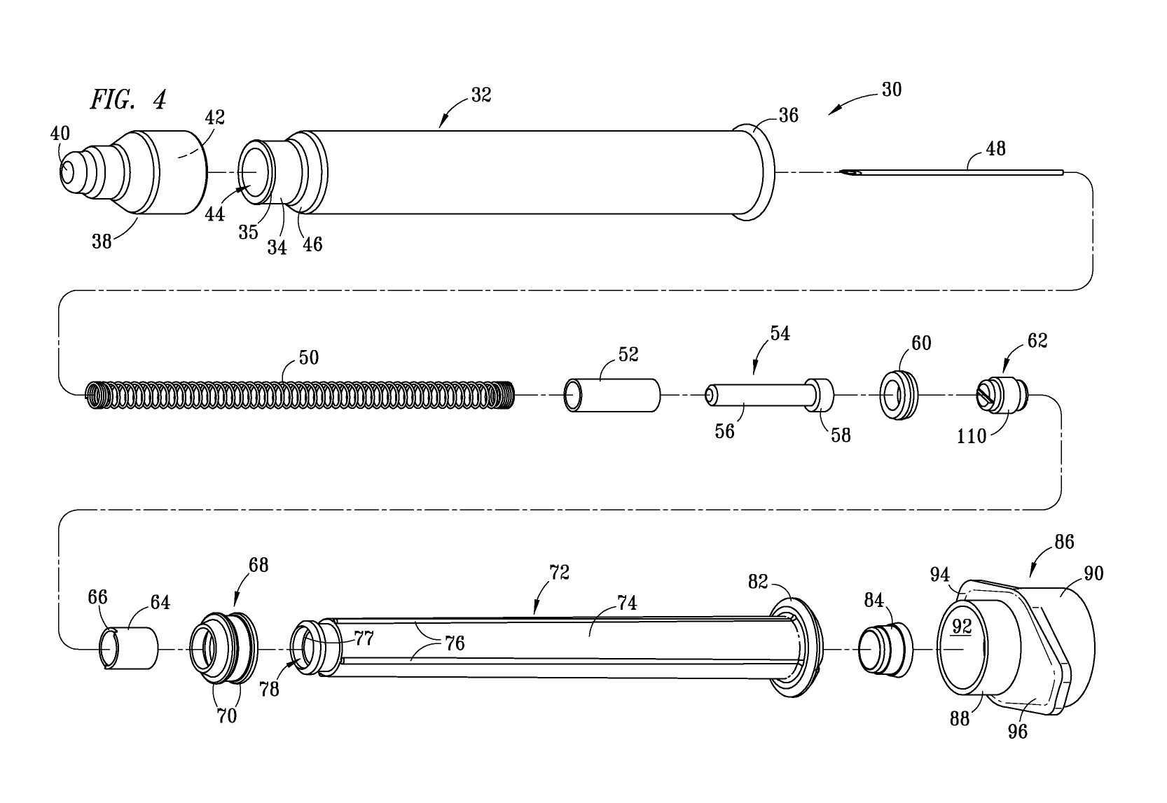

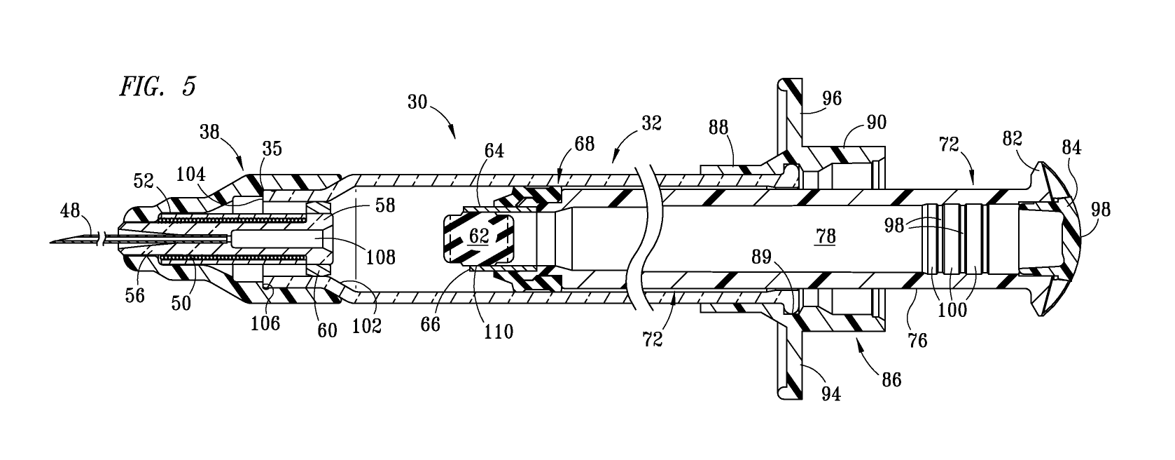

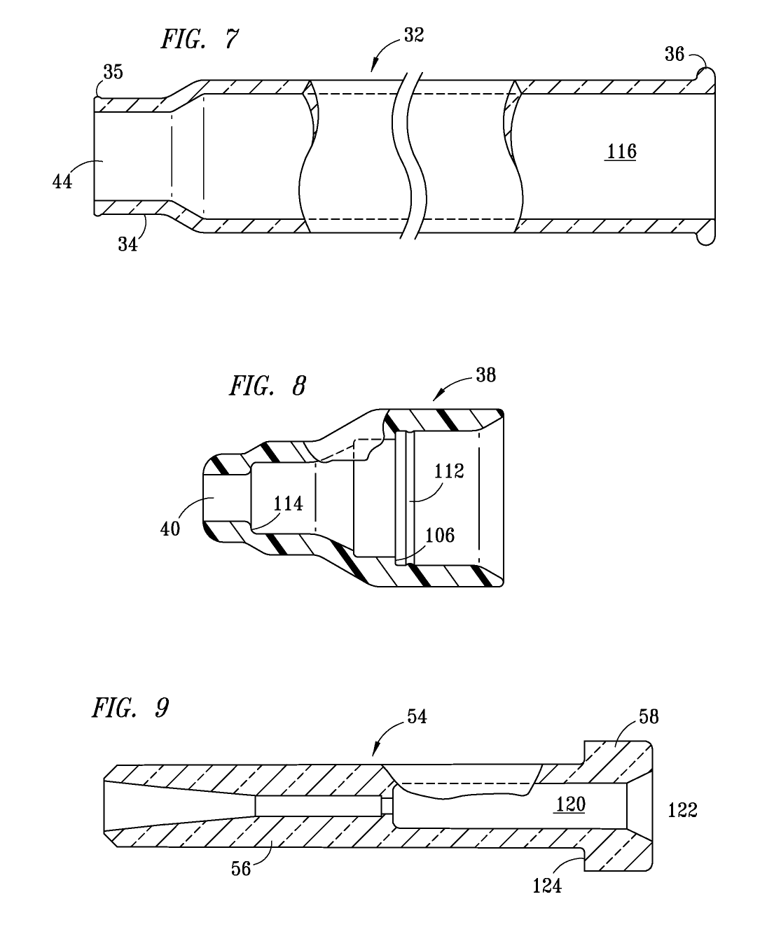

The component parts of syringe 30 are further described and explained in relation to FIGS. 4-5 and other Figures as mentioned below. Referring to FIGS. 4-6 and 7, barrel 32 is desirably made of glass, which is known to be substantially impermeable to gases and non-reactive with most liquids, and more particularly, non-reactive with therapeutic liquids with which syringes 30 are used. Barrel 32 preferably has a stepped tubular bore 44 with forwardly and rearwardly facing open ends, and further comprises a curved, outwardly projecting annular flange 36 disposed around the rearwardly facing open end. The forwardly facing end of barrel 32 preferably further comprises a smaller-diameter section having a tapered annular shoulder 46, a substantially cylindrical collar 34, and a slightly projecting annular flange 35 at the forward end of collar 34.

Barrel tip cap 38, as shown in FIGS. 4-6 and 8, is disposed forwardly of barrel 32, is preferably made of plastic, and comprises a stepped cylindrical bore having a small diameter, forwardly facing opening 40 through which part of needle holder 54 and needle 48 project when syringe 30 is assembled as shown in FIG. 5. Barrel tip cap 38 also has a larger diameter, rearwardly facing opening 42 that is designed to accommodate the outside diameter of recessed collar 34 of barrel 32. Barrel tip cap 38 can be attached to barrel 32 in various ways, such as by friction, snap-fit, or using an adhesive, or in any other known manner or using an attachment device that may become evident to one of ordinary skill in the art upon reading this disclosure. One preferred attachment method is frictional engagement between barrel tip cap 38 and barrel 32, which can be enhanced by slightly stretching open end 42 over the slightly projecting annular flange 35 at the front of barrel 32. If desired, a cooperating annular recess can be provided inside barrel tip cap 38 as described below in relation to FIG. 8. Another preferred attachment method is using a suitable commercially available adhesive to secure the inside surface of larger diameter opening 42 to the outside surface of cylindrical collar 34 during assembly of syringe 30. Such adhesive can also be applied following frictional attachment, for example, by applying glue in circumferentially extending, outwardly facing groove 37, as shown in FIG. 5. The attachment between barrel tip cap 38 and barrel 32 should be sufficiently strong to prevent barrel tip cap 38 from separating from barrel 32 when subjected to the forces exerted by compressible spring 50 when assembled as shown in FIG. 5, and when combined with any hydraulic forces experienced during use of syringe 32 together with the triggering or release forces required to initiate retraction.

If desired, barrel tip cap 38 can instead be made as a slide-on, snap-down piece; in two pieces that snap together around the front end of the barrel, or like a clamshell, having a foldable hinge that allows the cap to be applied from the side rather than from the front, which can be helpful if the needle is already projecting forwardly from the needle holder at the time the cap is installed.

FIGS. 4-6 further depict one embodiment of a retraction mechanism comprising retractable needle 48, compressible spring 50, sleeve 52, needle holder 54, and friction ring 60. Needle 48 is preferably a conventional metal needle having one beveled end, one blunt end and an internal fluid path between the two ends. Spring 50 is desirably made of any metal suitable for use in making compression springs of that size. Needle holder 54 is desirably made of glass, and is further described below. Sleeve 52, also depicted in FIGS. 21 and 22, is desirably made of metal, most preferably stainless steel, or of a suitable commercially available, durable plastic. Friction ring 60 is preferably made of elastomeric having a durometer appropriate to the size and spacing of the needle holder 54 and barrel 32, and adequate for achieving a fluid-tight seal when subjected to the operating pressures likely to be encountered during use of syringe 32.

Although needle 48 is shown in FIG. 4 as being exploded behind the rearwardly facing opening of barrel 32, the blunt end of needle 48 is desirably installed through opening 40 in the forwardly facing end of barrel tip cap 38 after barrel tip cap is attached to barrel 32 and after the other components of the retraction mechanism are installed through the rearwardly facing opening of barrel 32. Alternatively, needle 48 can be insert molded into needle holder 54 and installed from the rear, or, if using a suitably designed barrel tip cap, the friction ring, needle, needle holder, and spring can all be installed from the front if a holding structure or tool is provided to maintain the spring in compression during installation of the barrel tip cap.

Referring to the embodiment of FIGS. 4-6 and 9, needle holder 54 is preferably made of glass and has an elongated cylindrical front section 56 and a substantially shorter rear section 58 having an outside diameter greater than that of the front section. Referring to FIG. 9, needle holder 54 of this embodiment preferably further comprises a tapered throat 55 at the front end to guide the blunt end of needle 48 (FIG. 5) into position where it abuts against forwardly facing annular shoulder 55, establishing fluid communication between the inside of the needle and cylindrical cavity 120 through cylindrical throat 59.

Referring again to FIG. 5, the width of tapered throat 55 also provides a site around needle 48 where an adhesive can be applied to hold needle 48 in fixed axial relation to needle holder 54 when the adhesive is cured. The inside diameter of spring 50 is desirably large enough to slide over elongated section 56 of needle holder 54, but small enough to abut against the forwardly facing annular shoulder 124 of larger-diameter head portion 58 of needle holder 54. The outer diameter of spring 50 is desirably small enough to fit inside and be compressed inside bore 130 of sleeve 52 between ends 126, 128 (FIGS. 21-22), as shown in FIG. 5. Sleeve 52 acts as a spring guide during compression of spring 50, lessening any tendency of the spring to buckle during compression in the section where the inside diameter of barrel tip cap 38 widens.

Referring to FIGS. 4-6, friction ring 60 is desirably disposed around the outside diameter of head portion 58 of needle holder 54 and in frictional engagement with it. The outwardly facing surface of friction ring 60 desirably provides sealing engagement with the glass inside wall of barrel 32, and provides sufficient holding force on needle holder 54 to resist the spring force exerted by spring 50 in addition to any rearwardly directed pressure exerted on the front of needle 48 when needle 48 is inserted into a person or object during use. Because any liquid disposed inside the variable volume fluid chamber inside syringe 30 will contact part of friction ring 60, the friction ring is preferably made of an elastomeric material that is approved or suitable for use with such liquids and has a durometer within a suitable range for the size and type of syringe, spring, needle holder, etc. that are being used.

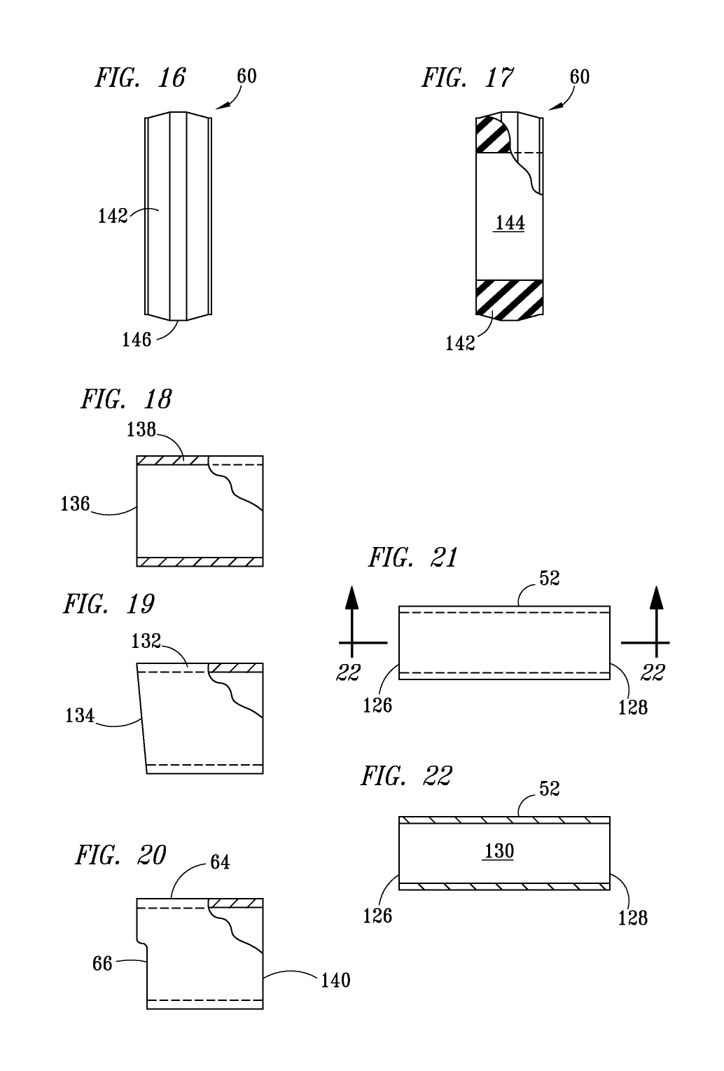

Referring to FIGS. 16 and 17, the friction ring 60 disclosed for use in this embodiment of syringe 30 preferably further comprises a generally toroidal structure having a cylindrical bore 144 and a circumferentially extending, outwardly facing surface comprising oppositely inclined surfaces 142, 143 joined along their lines of maximum diameter by a flatter outer band 146 having a width approximately equal to the width of each of the inclined sections. It will be appreciated, however, that other friction ring configurations can be similarly utilized to provide sealing engagement between the needle holder and the opposed wall of the barrel or other fluid-containment structure prior to retraction.

When assembled as shown in FIG. 5, spring 50 and tubular sleeve 52 are desirably seated between an annular shoulder 114 (FIG. 8) inside barrel tip cap 40 and the forwardly facing annular shoulder 124 of head 58 of needle holder 54 as shown in FIG. 9. After spring 50, sleeve 52, needle holder 54 and friction ring 60 are installed as shown in FIG. 5, the blunt end of needle 48 is desirably inserted into the forwardly extending open end of needle holder 54 as seen in FIGS. 5 and 9, and secured in place. The outside diameter of elongated section 56 of needle holder 54 is desirably slightly less than the inside diameter of forwardly facing opening 40 of barrel tip cap 38 to avoid undesirable flexing of needle 48 and to prevent spring 50 from jamming downwardly into any resultant annular space between the needle holder and tip cap. It will be appreciated by those of skill in the art upon reading this disclosure that barrel tip cap 38 can be unitarily molded with a cylindrical pedestal positioned and configured similarly to spring guide 52 in FIG. 5, in which case sleeve 52 is no longer needed. Such a pedestal could be made thicker except for a top portion that should have an outside diameter approximately equal to that of the larger-diameter section 58 of needle holder 54. Such a pedestal would still serve as a spring guide for spring 50 and could receive and support friction ring 60 away from spring 50 as friction ring 60 is forced forwardly and out of contact with needle holder 54 during retraction as discussed below.

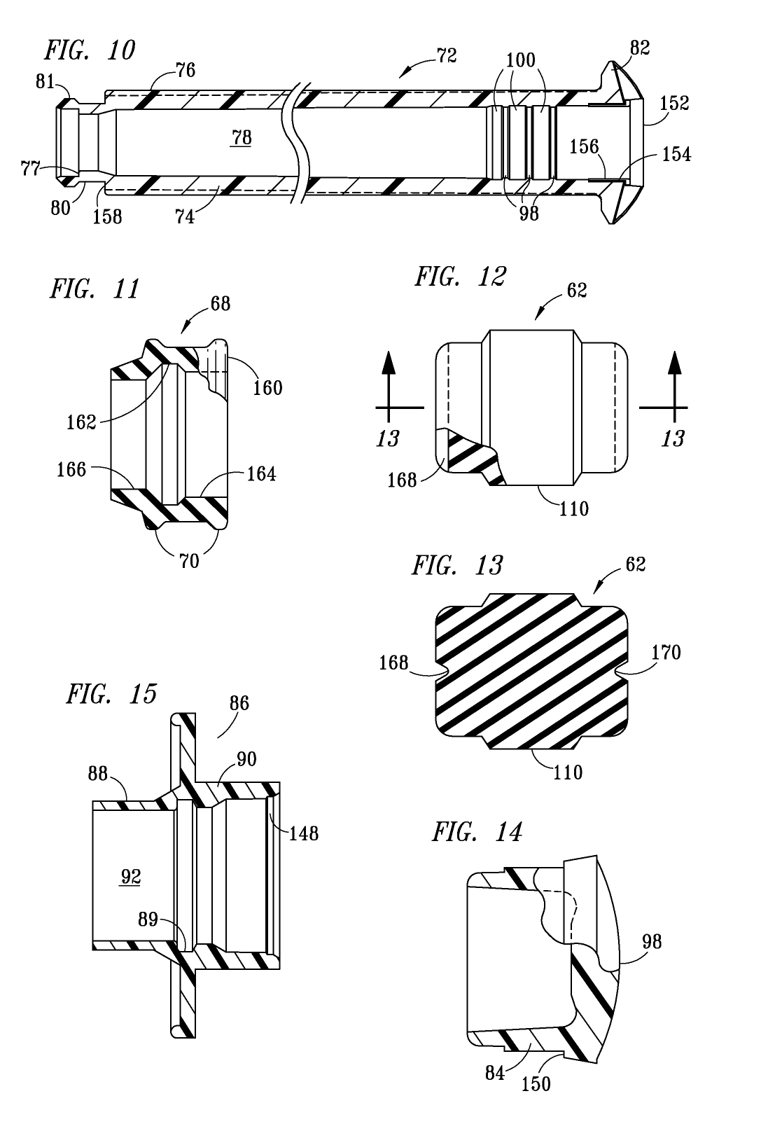

Referring to FIGS. 4-6, 10-14 and 18-20, plunger 72 preferably comprises elongated tubular handle 74, elastomeric plunger seal 68 disposed on the front end of handle 74, plunger sleeve 64 slidably insertable inside the open front end of plunger handle 74 and plunger seal 68, and extending forwardly therefrom (FIG. 5), elastomeric plunger plug 62, and a plunger end cap 98 disposed inside rear opening 152 of plunger handle 74. Referring particularly to FIG. 10, elongated tubular plunger handle 74 is preferably made of plastic and has forwardly and rearwardly facing openings and a retraction cavity 78 disposed between them, and further comprises a plurality of circumferentially spaced, substantially parallel, longitudinally extending ribs 76 that contact the inside wall of glass barrel 32 when plunger 72 is inserted in the barrel. Plunger handle 74 preferably further comprises a forwardly projecting annular flange 81 comprising an internal, forwardly facing annular shoulder 77 that abuts against the rear end 140 of plunger sleeve 64 (FIG. 20) when plunger sleeve 64 is inserted into the forwardly facing open end of plunger handle 74. Annular recess 80, disposed behind annular flange 81, cooperates with flange 81 to receive and support elastomeric plunger seal 68 in fixed axial relation to plunger handle 74.

Referring to FIGS. 10-11, an elastomeric plunger seal 68 is desirably seated in fixed axial relation around the forwardly facing opening of plunger handle 74, and has a plurality of axially spaced, circumferentially extending ribs 70 that provide fluid-tight sealing engagement with the inside wall of barrel 32 whenever the forwardly facing end of plunger 72 is inserted into sliding engagement with barrel 32. Plunger seal 68 preferably further comprises an internal annular recess 162 that seats on annular flange 81 of plunger handle 74 (FIG. 10). Plunger seal 68 is installed by pushing it over the front end of plunger handle 74 until rearwardly facing annular surface 160 of plunger seal 68 abuts against forwardly facing annular shoulder 158 (FIG. 10) of plunger handle 74 during the assembly of plunger 72. When plunger seal 68 is installed on plunger handle 74, annular inside surface 164 of plunger seal 68 seats inside recess 80, and annular inside surface 166 of plunger seal 68 is available to receive and frictionally engage plunger sleeve 64. When plunger sleeve 64 is inserted into engagement with annular inside surface 166 of plunger seal 68, sleeve 64 is desirably pressed inwardly until rearwardly facing end surface 140 abuts against forwardly facing annular shoulder 77 disposed inside flange 81 of plunger handle 74.

Referring again to FIG. 5, plunger sleeve 64 cooperates with elastomeric plunger seal 68 and plunger plug 62 (FIGS. 12-13, discussed below) to prevent any fluid disposed inside syringe 32 ahead of plunger 72 during manufacture, storage or use from contacting any plastic portion of plunger handle 74. Plunger sleeve 64 preferably has a stepped (FIG. 20) leading edge 66 for reasons discussed below in relation to retraction of needle 48, and is preferably seated inside the forwardly facing opening of plunger seal 68 and plunger handle 74 to facilitate the placement, seating and support of elastomeric plunger plug 62 so that plunger plug 62 seals any liquid or gaseous fluid disposed ahead of plunger 72 inside barrel 32 of syringe 30 out of retraction cavity 78 prior to retraction. Plunger plug 62 also cooperates with elastomeric plunger seal 68 and metal sleeve 64 to prevent liquid disposed inside syringe 32 from contacting any plastic surface. Although the plunger plug described in relation to the preferred embodiments is depicted as extending forwardly of the front end of the plunger handle, it will be appreciated upon reading this disclosure that a syringe with a plunger plug disposed with its forwardly facing end substantially flush with the front of the plunger handle can also function within the scope of the invention.

Referring to FIGS. 12 and 13, plunger plug 62 most preferably comprises a symmetrically formed, solid elastomeric body having reduced-diameter sections 170 with chamfered ends and transverse diametrical slots 168 disposed at each end to facilitate the insertion of either end into the opening at the front of plunger handle 74. A centrally disposed section 110 having a larger diameter is desirably provided in plug 62 to reduce the length of the portion of plug 62 that frictionally engages the inside of plunger handle 74 without simultaneously reducing the overall length of plug 62, thereby reducing the length of plug travel that is required to dislodge plug 62 from the forwardly facing plunger opening during retraction.

Referring to FIGS. 4-6, 10 and 14, the rearwardly facing opening of plunger handle 74 is desirably sized to receive plunger end cap 84 into seated frictional engagement, thereby sealing the rear of retraction cavity 78 except for air vents 156, 154, 82 that are desirably provided in or around plunger end cap 84 to eliminate or reduce any likelihood that some portion of liquid will be forcibly expelled from the font of syringe 32 as plunger plug 62 and needle holder 54 are propelled rearwardly into retraction cavity 78 by spring 50 during retraction. Because plunger seal 68 and plunger plug 62 are made of rubbery elastomers and not plastic, the combined use of plunger seal 68, metal sleeve 64 and plunger plug 62 at the front of plunger handle 74 prevents any liquid disposed inside syringe 30 from contacting any portion of plastic plunger handle 74 prior to retraction. Referring to FIGS. 5 and 14, plunger end cap 84 is preferably made of plastic, and further comprises a rearwardly facing surface 98 against which thumb pressure can be applied by the user while supporting and controlling barrel 32 with finger pressure applied to the forwardly facing surfaces of finger grips 94, 96, as discussed in greater detail below.

Although barrel grip 86 is shown in FIG. 4 as being exploded rearwardly behind plunger 32, it should be understood that barrel grip 86 as disclosed in FIGS. 4-5, 6 and 15 is preferably installed onto barrel 32 from the front of barrel 32 either prior to the attachment of barrel tip cap 38 to barrel 32, or at least prior to the installation of needle 48. Referring to FIGS. 4 and 15, circumferentially extending flange 36 at the rear of barrel 32 is most easily seated in cooperative annular recess 89 of barrel grip 86 if barrel grip 86 is pushed onto barrel 32 from the forwardly facing end. When barrel grip 86 is seated in place around the rearwardly facing end of barrel 32, manual force can be applied to oppositely disposed, outwardly projecting finger grips 94, 96 of barrel grip 86 without experiencing axial slippage between barrel grip 86 and barrel 32. Forwardly extending annular collar 88 of barrel grip 86 also assists in maintaining stability between barrel grip 86 and barrel 32. To achieve this benefit, however, inside diameter 92 of collar 88 should be only slightly larger than the outside diameter of barrel 32 just ahead of flange 36.

Once plunger 72 is assembled as described herein and barrel grip 86 has been installed around the rearwardly facing end of barrel 32, the forwardly facing end of plunger 72 can be inserted through rear collar 90 of barrel grip 86 into the rearwardly facing opening of barrel 32. Once needle 48 has been installed in the front part of needle holder 54, the assembled syringe 30 will have the configuration depicted in FIG. 5. Following application of a needle cover to barrel tip cap 38, the assembly is complete, and syringe 30 can be packaged and sterilized. Where syringe 30 is intended for use as a prefill, however, a therapeutic liquid is desirably loaded into barrel 32 through its rearwardly facing opening prior to insertion of plunger 72. In a prefilled application, a plug can be provided inside the needle cap to seal the tip of the needle prior to use. The glass inside wall of barrel 32 cooperates with the elastomeric and metal surfaces disposed at the forwardly facing end of plunger 72 and with the glass needle holder 54 and the rearwardly facing surface of elastomeric friction ring 60 to insure that no liquid disposed in the variable volume liquid containment chamber thus formed can contact any plastic surface prior to use. Also, the disclosed syringe 30 does not embody any glass element that directly contacts any other glass element during assembly, storage or use.

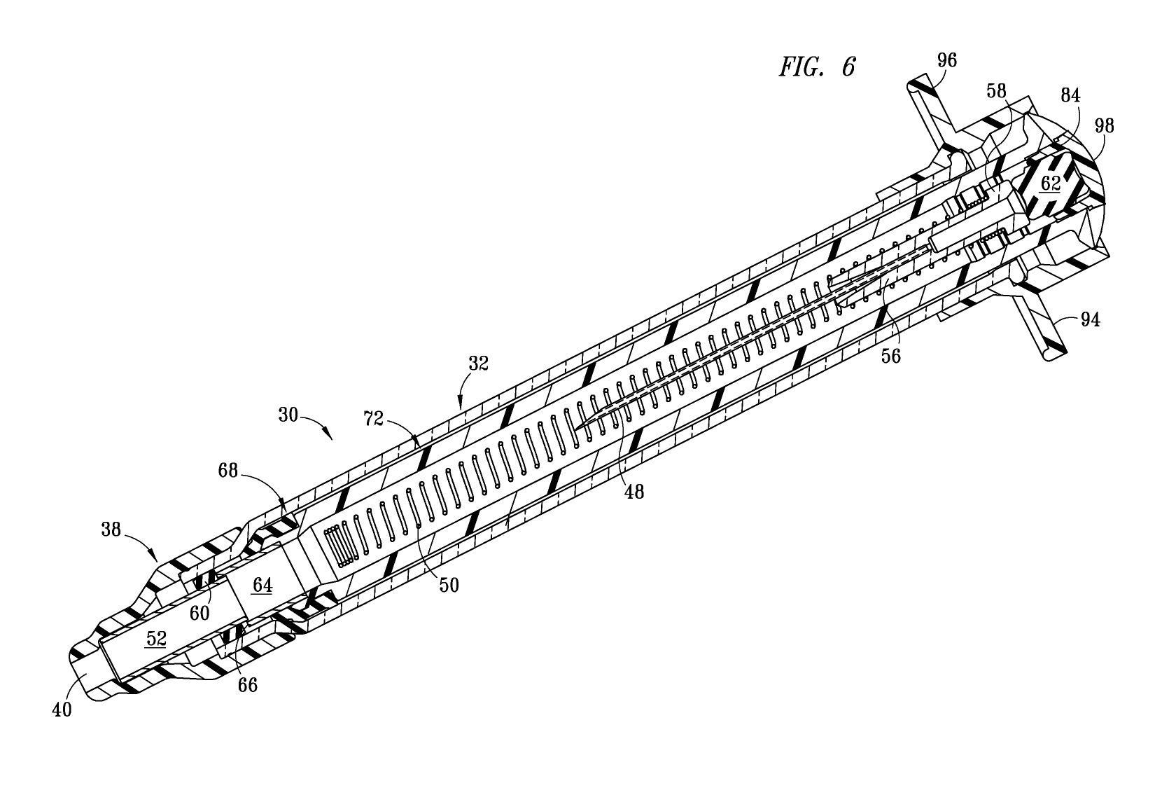

Referring to FIGS. 5-6, following discharge of substantially all the liquid from barrel 32 through needle 48 during use of syringe 30, the continued application of thumb pressure to the rear of plunger 72 while holding barrel 32 in a substantially fixed position using finger grips 94, 96 will cause the forwardly facing end of plug 62 to abut the rearwardly facing annular surface of large-diameter section 58 of needle holder 54, causing plunger plug 62 to slide rearwardly to a point where friction ring 60 is contacted by leading edge 66 of plunger sleeve 64. Continued pressure exerted against the rear of plunger 74 will cause leading edge 66 of plunger sleeve 64 to force friction ring 60 forwardly to a position where the annular inside surface of friction ring 60 moves onto outside surface of sleeve 52, at which point friction ring 60 is no longer holding the large-diameter portion 58 of needle holder 54 against the force of compressed spring 50. Continued pressure exerted on the back of plunger 72 relative to barrel grip 86 and barrel 32 will cause the rearwardly facing end of needle holder 54 to push plunger plug 62 rearwardly through plunger sleeve 64 until larger-diameter, centrally disposed section 110 of plunger plug 62 begins to disengage from the inside wall of plunger handle 74 at the point where retraction cavity 78 widens. As the surface area of section 110 contacting the inside wall of plunger handle 74 is reduced, a point is reached where the force of spring 50 surpasses the remaining frictional holding force of plug 62, at which point spring 50 drives needle holder 54 and plunger plug 62 rearwardly through retraction cavity 78 until plunger plug 62 contacts the underside of plunger end cap 84 as seen in FIG. 6. As elongated section 56 of needle holder 54 (FIG. 4) is propelled rearwardly through retraction cavity 78, it carries needle 48 rearwardly as well, thereby retracting the beveled end of needle 48 inside syringe 30, rendering syringe 30 safe for subsequent handling and disposal, and effectively preventing its reuse. In the fully retracted position shown in FIG. 6, friction ring 60 remains attached to the outside of sleeve 52, and the peripheral edge of the rear of plunger handle 74 is lodged inside rearwardly facing collar 90 (FIG. 5) of barrel grip 86.

When plunger sleeve 64 contacts friction ring 60 as described above, the use of a stepped front or leading edge 66 as shown in FIG. 20 is believed to reduce the thumb force required to initiate retraction by initially concentrating the force on one side of friction ring 60, thereby overcoming inertia and initiating movement of one side of the ring relative to section 58 of the needle holder just prior to contacting the other side. Another embodiment of a plunger sleeve useful for achieving this purpose is illustrated in FIG. 19 by inclined leading edge 134 of plunger sleeve 132. Also, even though it is less preferred and although a greater thumb force will likely be required to initiate retraction, it will be appreciated that a cylindrical plunger sleeve 138 having a perpendicularly transverse leading edge 136 can also be used within the scope of the invention if desired.

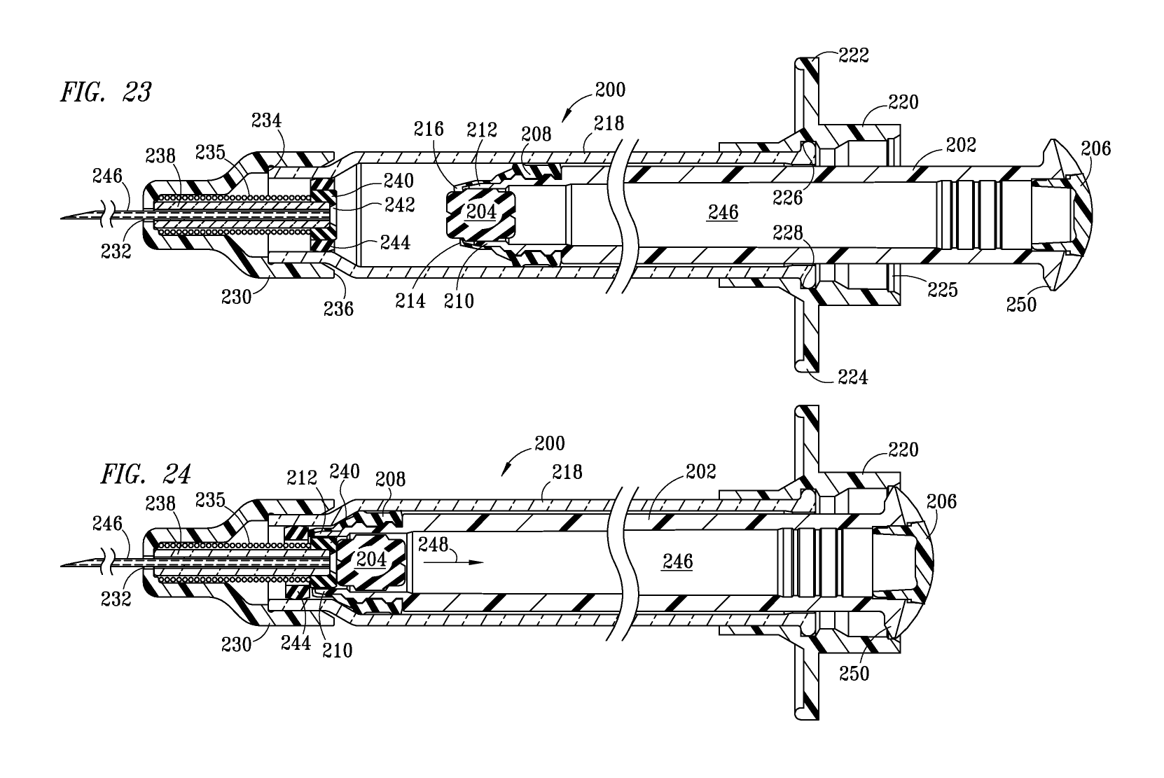

Another embodiment of the invention is disclosed and described in relation to FIGS. 23-25. This embodiment does not require the use of either sleeve 52 or plunger sleeve 64 as disclosed above, which reduces the total number of parts in the syringe and also reduces the number of metal parts needed to prevent any liquid-to-plastic contact or any glass-to-glass contact inside a syringe having a glass barrel and a retractable needle. These benefits are achieved within a design structure that is very similar to that disclosed and described in relation to FIGS. 1-22 above. One principal difference is the use of needle holder comprising a glass tube having a substantially constant outside diameter over its entire length in combination with an elastomeric needle head cap having a centrally disposed opening to allow fluid communication between the variable volume chamber inside the syringe and the fluid pathway through the needle holder and needle. A needle holder cap 240 can be axially positioned relative to the glass tube of the needle holder by an annular flange extending radially inward behind the glass tube or by any other similarly effective means. In this embodiment, friction ring 244 is preferably a separate piece, but alternatively, one could fabricate needle holder cap 240 and friction ring 244 unitarily with a continuous web between them, and provide a cutter or other separation device above or below the web to aid in separating cap 240 and ring 244 during retraction. Another principal difference in the preferred embodiment is the use of a forwardly elongated plunger seal that wraps over the forwardly extending end of the plunger handle to prevent any liquid-to-plastic contact prior to retraction.

Referring to FIGS. 23-25, syringe 200 preferably comprises glass barrel 218 made substantially the same as barrel 32 previously disclosed, that has a barrel grip 220, preferably made of plastic, attached to its rear end and a barrel tip cap, also preferably made of plastic, attached to its forwardly facing end by recess 228 that engages circumferentially extending flange 226 at the rear of barrel 218. Plunger handle 202 is inserted into barrel 218 through the rearwardly facing opening in barrel 218 and barrel grip 220, and has a plunger seal 208 disposed in fixed axial relation to plunger handle 202 that provides a slidable, fluid-tight seal with the inside wall of barrel 218 and blocks front section 212 of plunger handle 202 from contacting any liquid disposed inside syringe 200 during use. Plunger 202 preferably further comprises plunger plug 204 lodged inside an opening in the front of retraction cavity 246 and a plunger end cap 206 engaging a rearwardly facing opening in flange 250 at the rear of retraction cavity 246.

The retraction mechanism of syringe 200 preferably comprises glass tubular needle holder 238 with attached needle 246, compressed metal spring 235, elastomeric needle holder cap 240, and elastomeric friction ring 244. The beveled end of needle 246 extends forwardly through aperture 232 in barrel tip cover 230 that has a diameter sufficiently small to retain the forwardly facing end of needle holder 238 behind it. Compressed spring 235 is disposed around tubular needle holder 238, with the forwardly extending portion being disposed in an annular space between barrel tip cap 230 and needle holder 238.

During retraction, forwardly extending portion 216 of plunger seal 208, backed by forwardly extending annular tip 212 of plunger handle 202, contacts and moves friction ring 244 forward relative to needle holder cap 240, to a position where needle cap holder 240 is released and spring 235 can begin to expand rearwardly. Although the plunger plug described in relation to the preferred embodiments is depicted as extending forwardly of the front end of the plunger handle, it will be appreciated upon reading this disclosure that a syringe with a plunger plug disposed with its forwardly facing end substantially flush with the front of the plunger handle can also function within the scope of the invention, as the plug does not have to contact the needle holder first in every embodiment of the invention.

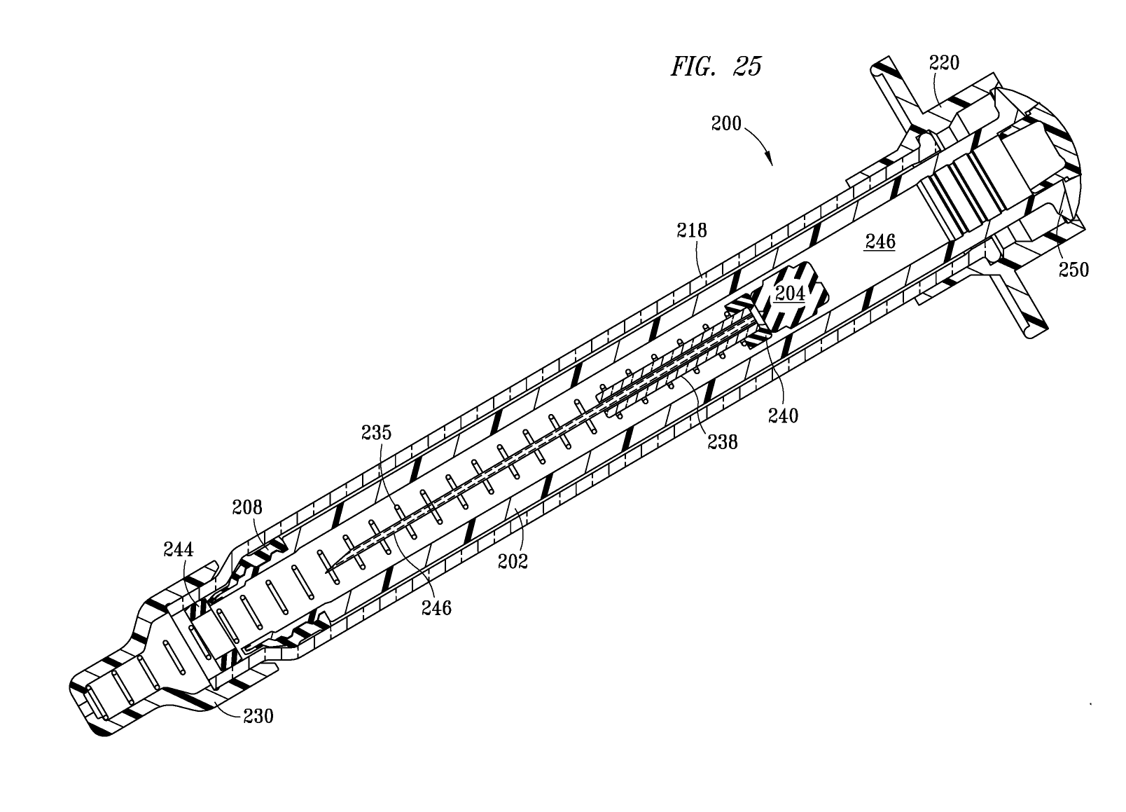

As friction ring 244 moves forwardly, plunger plug 204 is moved rearwardly to a wider-diameter section of retraction cavity 246. Once friction ring 244 is released from needle holder cap 240 and plunger plug 204 is dislodged from annular tip 212 of plunger handle 202, needle holder 238, needle 246, needle holder cap 240 and plunger plug 204 are all driven rearwardly by the force of spring 235, as indicated by arrow 248 (FIG. 24). Following retraction, as shown in FIG. 25, needle 246 is fully contained inside barrel 218 and syringe 200 is rendered safe and unusable.

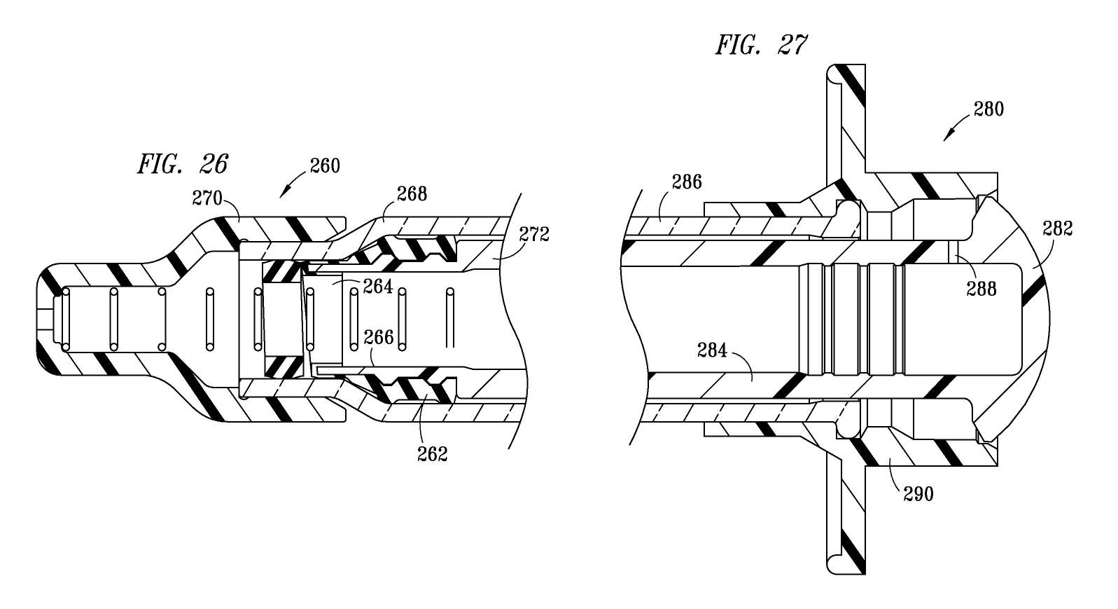

Referring to FIG. 26, another embodiment of the invention is disclosed wherein portion 264 of plunger seal 262 of syringe 260 is formed inside the front tip of plunger 272 and provides an engagement and seating surface for the plunger plug, which has already been displaced in this view from a position substantially as shown in FIGS. 23 and 24. With this embodiment of the invention, it is possible to eliminate one of the step-downs in the inside diameter of inside wall 266 of plunger 272 as compared, for example, to the structure shown in the embodiment of FIGS. 23-25.

Referring to FIG. 27, another embodiment of the invention is disclosed wherein plunger 284 of syringe 280 has a unitarily molded, closed end cap 282 and a vent aperture 288 disposed in plunger 284 forwardly of end cap 282.

Other alterations and modifications of the invention will likewise become apparent to those of ordinary skill in the art upon reading this specification in view of the accompanying drawings, and it is intended that the scope of the invention disclosed herein be limited only by the broadest interpretation of the appended claims to which the inventors are legally entitled.

1-14. (canceled)

15. A syringe useful for injecting a therapeutic fluid, the syringe comprising a barrel, a barrel tip cap attached to the barrel, a plunger that is forwardly moveable inside the barrel during injection, a needle that is retractable after injection from a first position in which the needle projects forwardly of the barrel tip cap to a second position in which the needle does not project forwardly of the barrel tip cap, and a needle retraction mechanism, the syringe having a plurality of liquid containment surfaces that define a variable volume chamber that confines the therapeutic fluid prior to injection and decreases in volume as the plunger is advanced forwardly during injection, the liquid containment surfaces comprising one or more materials that are substantially non-reactive with the therapeutic fluid and are suitable for use in a pharmaceutical grade application.

16. The syringe of claim 15 wherein at least one of the liquid containment surfaces is made of glass.

17. The syringe of claim 15 wherein at least one of the liquid containment surfaces is made of metal.

18. The syringe of claim 15 wherein at least one of the liquid containment surfaces is made of rubber.

19. The syringe of claim 15 wherein none of the liquid containment surfaces is made of plastic.

20. The syringe of claim 15 wherein the therapeutic fluid is a liquid.

21. The syringe of claim 15 wherein the therapeutic fluid comprises a liquid containing dispersed solids.

22. The syringe of claim 15 wherein the therapeutic fluid comprises a liquid containing dissolved or dispersed gas.

23. The syringe of claim 16 wherein no liquid containment surface that is made of glass contacts another liquid containment surface that is made of glass.

24. The syringe of claim 15 wherein the needle retraction mechanism comprises a needle holder, a retraction spring that biases the needle holder rearwardly relative to the barrel, and a holding member that resists rearward movement of the needle prior to and during the injection.

25. The syringe of claim 24 wherein the holding member comprises an elastomeric portion.

26. The syringe of claim 24 wherein at least part of the holding member surrounds at least a portion of the needle holder.

27. The syringe of claim 15 comprising a flow path for the therapeutic fluid that does not allow contact between the therapeutic fluid and a plastic surface prior to retraction of the needle.

28. The syringe of claim 15 having sufficient structural integrity to withstand any fluid pressures encountered during manufacture, use or storage of the syringe.

29. The syringe of claim 15 wherein all surfaces that are wetted by the therapeutic fluid during storage or use of the syringe prior to retraction are made of a material that is inert to the therapeutic fluid.

30. The syringe of claim 15 in combination with a therapeutic fluid disposed inside the variable volume chamber.

31. The syringe of claim 24 wherein the barrel and needle holder are made of glass.

32. The syringe of claim 15 wherein the plunger comprises a plunger handle, plunger plug, plunger seal and plunger sleeve.

33. The syringe of claim 32 wherein the plunger plug, plunger seal and plunger sleeve comprise liquid containment surfaces that are not plastic.

34. The syringe of claim 24 wherein the plunger comprises a retraction cavity into which at least a portion of the needle and retraction spring are received during retraction.

35. The syringe of claim 30 that is prefilled with the therapeutic fluid during manufacture.

36. The syringe of claim 15 further comprising a barrel grip.

37. The syringe of claim 36 wherein the barrel grip further comprises outwardly projecting finger grips.

38. The syringe of claim 15 wherein the plunger further comprises a plunger end cap.

39. The syringe of claim 15 wherein the barrel tip cap comprises a stepped cylindrical bore.

40. The syringe of claim 15 wherein the barrel tip cap comprises a substantially cylindrical collar having a rear opening, a smaller diameter front opening, and an inwardly projecting annular flange near the front opening.

41. The syringe of claim 15 wherein the barrel tip cap is made of plastic.

42. The syringe of claim 32 wherein the plunger sleeve is made of stainless steel.

43. The syringe of claim 40 wherein the barrel has a forwardly extending recessed collar that is inserted inside the cylindrical collar of the barrel tip cap.

44. A syringe having a rearwardly biased retractable needle and wetted fluid containment surfaces that are made of a material other than plastic.

45. A syringe as in claim 44 that is prefilled with a therapeutic fluid.

46. The syringe of claim 45 in which the therapeutic fluid comprises solids dissolved in a liquid.

47. The syringe of claim 45 in which the therapeutic fluid comprises gas dissolved in a liquid.

48. The syringe of claim 45 wherein the fluid containment surfaces are made of a material that is not susceptible to leaching when contacted with the therapeutic fluid.

49. The syringe of clam 45 wherein the fluid containment surfaces are made of a material that is not susceptible to migration of the therapeutic fluid through the material.

50. The syringe of claim 44 wherein the material other than plastic is selected from the group consisting of glass, metal and elastomers.

51. The syringe of claim 50 wherein there is no glass-to-glass contact.

52. A prefilled syringe having a retractable needle, the syringe comprising a liquid flow path with all wetted surfaces made of a material or materials approved for pharmaceutical grade applications.

53. The syringe of claim 52 configured to withstand all pressures to which the syringe is subjected during normal operation.

54. The syringe of claim 52 comprising a needle holder having at least some portion made of glass.

55. The syringe of claim 52 having no parts in glass-to-glass contact.

56. The syringe of claim 52 having wetted internal surfaces that are inert to a therapeutic fluid disposed inside the syringe.

57. A syringe comprising:
a barrel having a glass inside wall and a tip cap disposed in fixed relation around a forwardly facing end of the barrel;
a plunger slidably disposed inside the barrel, the plunger having a plunger handle with a forwardly facing end, an elastomeric plunger seal disposed around the forwardly facing end of the plunger handle and also contacting the inside wall of the barrel, and an elastomeric plunger plug releasably disposed inside an opening in the forwardly facing end of the plunger handle;
a needle;
and a needle retraction mechanism comprising a glass needle holder having a rearwardly facing end, an elastomeric needle holder cap surrounding the rearwardly facing end of the needle holder and providing a liquid seal between the needle holder and the needle holder cap, and a holding member surrounding the needle holder cap, the holding member comprising an elastomeric ring that also contacts and provides a liquid seal between the inside wall of the barrel and the needle holder cap;
wherein the needle cooperates with the needle holder, the needle holder cap, the elastomeric ring, the inside wall of the barrel, the plunger plug and the plunger seal to define a fluid containment chamber of variable volume, and
wherein a liquid disposed inside the fluid containment chamber does not directly contact any plastic surface prior to use of the syringe.

58. A syringe comprising:
a barrel having a centrally disposed glass tubular section and forwardly and rearwardly disposed open ends;
a plastic barrel grip attachable to the rearwardly disposed end of the barrel, the barrel grip comprising a longitudinal bore engageable with the barrel and a plurality of oppositely disposed finger grips;
a plunger insertable through the rearwardly disposed open end of the barrel and having a forwardly extending portion that is slidably disposed inside the barrel;
and a plastic barrel tip cap attached in fixed relation around the forwardly disposed open end of the barrel, the barrel tip cap also having a centrally disposed, forwardly facing aperture through which a needle projects, the needle being retractable inside the barrel following use of the syringe.

59. A syringe useful for injecting a therapeutic fluid, the syringe comprising a barrel, a plunger that is forwardly moveable inside the barrel during injection, a needle that is retractable after injection from a first position in which the needle projects forwardly of the barrel to a second position in which the needle does not project forwardly of the barrel, and a needle retraction mechanism, the syringe having a plurality of liquid containment surfaces that define a variable volume chamber that confines the therapeutic fluid prior to injection and decreases in volume as the plunger is advanced forwardly during injection, the liquid containment surfaces comprising one or more materials that are substantially non-reactive with the therapeutic fluid and are suitable for use in a pharmaceutical grade application.
